(19) United States
(12) Patent Application Publication
(10) Pub. No.: US 2014/0121164 A1
(43) Pub. Date: May 1, 2014

(54) PREVENTION OF KIDNEY INJURY OR DISEASE

(71) Applicants: University of Florida Research Foundation, Gainesville, FL (US); ABBVIE, INC., NORTH CHICAGO, IL (US)

(72) Inventors: SOREN NIELSEN, AABYHOEJ (DK); THOMAS ENGELBRECHT NORDKILD JONASSEN, HOLTE (DK); SAMINA KHAN, HIGHLAND PARK, IL (US); MARK T. HOUSER, LAKE FOREST, IL (US); IB BO LUMHOLTZ, CHARLOTTENLUND (DK); MICHAEL BECKERT, BERLIN (DE); THOMAS BEAVER, GAINESVILLE, FL (US)

(21) Appl. No.: 14/069,045

(22) Filed: Oct. 31, 2013

Related U.S. Application Data
(63) Continuation of application No. 13/923,193, filed on Jun. 20, 2013.
(60) Provisional application No. 61/739,183, filed on Dec. 19, 2012.
(60) Provisional application No. 61/725,873, filed on Nov. 13, 2012.
(60) Provisional application No. 61/721,371, filed on Nov. 1, 2012.

(51) Int. Cl. A61K 38/10

(52) U.S. Cl. CPC A61K 38/10
USPC 514/10.7

(57) ABSTRACT

The present invention relates to a dosage regime of a peptide analogues of [alpha]-melanocyte-stimulating hormone ([alpha]-MSH), which possesses an increased efficacy compared to the native [alpha]-MSH peptide in the treatment or prevention of kidney injury or disease.

### CROSS REFERENCE TO RELATED APPLICATIONS

This application claims priority to U.S. Provisional Application No. 61/739,183, filed Dec. 19, 2012; U.S. Provisional Application No. 61/725,873, filed Nov. 13, 2012; U.S. Provisional Application No. 61/721,371, filed Nov. 1, 2012; and U.S. Provisional Application No. 61/710,972, filed Oct. 8, 2012.

This application incorporates by reference the Sequence Listing filed herewith and entitled “SEQ DATA_ST25”, which was created on Oct. 8, 2012 and has a size of 1 KB.

### TECHNICAL FIELD OF THE INVENTION

The present invention relates to dosing regimens for treating or preventing kidney injury or disease.

### BACKGROUND OF THE INVENTION

Acute Kidney Injury (AKI)/acute renal failure (ARF)—is a rapid, sometimes reversible, kidney injury which can lead to increased mortality and co-morbidity. More than 500,000 patients in the USA and the EU each year undergo major cardiac surgery, and a significant fraction develops kidney injury, such as AKI resulting in prolonged hospitalization or even death.

Melanocortin receptor (MCr) agonists have shown marked immune modulating and organ protective effects in animal disease models, including sepsis- and surgery-induced.

AP214 (SEQ ID NO: 1, which is further N-terminally acetylated and C-terminally amidated) is a novel non-selective MCr agonist. Matthew N. Simmons et al. has shown that AP214 provides a renoprotective effect in pigs after induction of complete warm ischemia in the kidney. All AP214 in that study was done at 200 μg/kg and injected as a 10 ml bolus intravenously during 1 minute. Seven AP214 doses were administered, including 10 minutes before unclamping, 3 hours after unclamping, on the morning of postoperative day 1, and 4 doses every 24 hours for an additional 4 days.

### SUMMARY OF THE INVENTION

It was unexpectedly discovered that instead of seven sequential doses as used in pigs, a minimal of three properly-timed, sequential doses would be sufficient to effectively treat or prevent AKI in humans who undergo cardiac or other major surgeries or medical procedures.

Thus, one aspect of the invention relates to a method of preventing or reducing Acute Kidney Injury (AKI) in a subject undergoing surgery comprising cross clamping, wherein the method comprises:
I. administering to the subject a first dosage of 150 μg/kg to 400 μg/kg bodyweight of a peptide comprising the amino acid sequence set forth in SEQ ID NO: 1 (e.g. AP214) or a pharmacologically acceptable salt thereof, said administration being initiated before said surgery (e.g., prior to or at skin incision);
administering to the subject a second dosage of 150 to 600 μg/kg bodyweight of said peptide or pharmacologically active salt thereof, administration of said second dosage being initiated prior to or at cross clamp release; and
III. administering to the subject a third dosage of 150 to 400 μg/kg bodyweight of said peptide or pharmacologically active salt thereof, administration of said third dosage being initiated 1-24 hours after cross clamp release (e.g., at 6 hours after cross clamp release).

In some embodiments, the method does not include any additional dosing of said peptide (e.g., AP214) or pharmacologically active salt thereof beyond 24 hours after cross clamp release.

Preferably, each administration or infusion of a peptide dosage (e.g., an AP214 dosage) lasts 5 minutes or longer. More preferably, each infusion or administration of a peptide dosage (e.g., an AP214 dosage) lasts 10 minutes or longer.

Also preferably, the surgery is a cardiovascular surgery.

The example section provides the experimental background for the selected treatment protocol according to the present invention.

### BRIEF DESCRIPTION OF THE FIGURES

FIG. 1 shows the effect of AP214 (600 μg/kg) and placebo on eGFR levels (1a) and on serum creatinine levels (1b).

FIG. 2 shows the effect of AP214 (600 μg/kg) and placebo on cystantine levels (2a) and carbamide levels (2b).

FIG. 3 shows the number of patients developing AKI in response to AP214 (600 μg/kg) and placebo indicated as a fraction of the total number of patients according to the AKIN score (3a) and the RIFLE score (3b).

FIG. 4 shows the study design of the CS007 trial.

FIG. 5 shows both short and long term efficacy signals in relation to AKI.

FIG. 6 shows that both the 600 and 800 μg/kg doses provides a reduction in negative outcomes for patients.

FIG. 7 shows significantly lower GFR change (reduction) at Day 90 compared to baseline for AP214 800 μg/kg dose vs. placebo.

The present invention will now be described in more detail in the following.

### DETAILED DESCRIPTION OF THE INVENTION

The present invention provides a dosage regime for the effective treatment, prevention or reduction of AKI or other kidney injury or disease in humans.

In a first aspect, the present invention relates to a method of preventing or reducing Acute Kidney Injury (AKI) in a subject undergoing surgery comprising cross clamping, the method comprises:
I. administering to the subject a first dosage of 150 μg/kg to 400 μg/kg bodyweight of a peptide comprising the amino add sequence set forth in SEQ ID: NO 1 (e.g., AP214) or a pharmacologically acceptable salt thereof, said administration being initiated before said surgery (e.g., prior to or at skin incision);
II. administering to the subject a second dosage of 150 to 600 μg/kg bodyweight of said peptide or pharmacologically active salt thereof, administration of said second dosage being initiated prior to or at cross clamp release; and
III. administering to the subject a third dosage of 150 to 400 μg/kg bodyweight of said peptide or pharmacologically active salt thereof, administration of said third dosage being initiated 1-24 hours after cross clamp release (e.g. at 6 hours after the second dosage).

The above provided dosage regime was unexpectedly found effective in the prevention or reduction of AKI in humans, even without any additional AP214 dosing after 24 hours post cross clamp release It is to be understood that the initiation of administration of the first, the second and/or the third dosage relates to initiation of infusion.

Preferably, said peptide or pharmacologically acceptable salt is 19 amino acid residues in length. The 19 amino acid peptide is the exact length of the tested peptide with SEQ ID NO: 1:

(SEQ ID NO: 1)
Lys-Lys-Lys-Lys-Lys-Lys-Ser-Tyr-Ser-Met-Glu-His-
Phe-Arg-Trp-Gly-Lys-Pro-Val.

More preferably, said peptide is SEQ ID NO: 1 which is further N-terminally acetylated and C-terminally amidated (i.e., AP214). Thus, the carboxy terminus of the peptide is modified by amidation. Thus, the invention relates to a peptide (SEQ ID NO:1), wherein the carboxy terminus of is —C(═O)—B1, wherein B1=NH₂. Similarly the amino terminus of the peptide is modified by acetylation. Thus, in the peptide (SEQ ID NO:1) the amino terminus is (B6)HN—, wherein B6=B4-C(═O)—, and B4=CH₃.

Thus, AP214 may also be described by: Acetyl-Lysyl-L-lysyl-L-lysyl-L-lysyl-L-lysyl-L-lysyl-L-seryl-L-tyrosyl-L-seryl-L-methionyl-Lglutamyl-L-histidyl-L-phenylalanyl-L-arginyl-L-tryptophanyl-glycyl-L-lysyl-L-prolyl-L-valine amide. Another name may be Acetyl-(Lys)₆-α-MSH.

A salt (e.g., an acetate salt) of AP214 can also be used in any method of the invention described or contemplated herein. Preferably, the peptide used in any method of the invention described or contemplated herein is AP214 acetate.

The different boluses of the dosage regime may be provided with equal or different concentrations. In an embodiment the dosages are administered with equal amounts or substantially equal amounts of said peptide or pharmacologically active salt.

The concentration of the first dosage may vary. Preferably, the first dosage is in the range of 150-300 μg of said peptide or pharmacologically active salt per kg bodyweight, such as in the range of 150-250 μg/kg bodyweight, such as in the range of 150-200 μg/kg bodyweight, such as in the range 200-300 μg/kg bodyweight. More preferably, the first dosage is 200 μg/kg bodyweight. Also more preferably, the first dosage is 300 μg/kg bodyweight.

Similar, the concentration of the second dosage may vary. Preferably, the second dosage is in the range of 200-600 μg of said peptide or pharmacologically active salt per kg bodyweight, such as in the range of 300-600 μg/kg bodyweight, such as in the range of 400-600 μg/kg bodyweight, such as in the range of 500-600 μg/kg bodyweight, such as in the range 200-300 μg/kg bodyweight, such as in the range of 200-400 μg/kg bodyweight, such as in the range of 200-500 μg/kg bodyweight, such as in the range 300-400 μg/kg bodyweight, such as in the range 300-500 μg/kg bodyweight, such as in the range 400-500 μg/kg bodyweight. More preferably, the second dosage is 400 μg/kg bodyweight. Also more preferably, the second dosage is 600 μg/kg bodyweight. The present invention also contemplates the use of more than 600 μg/kg bodyweight of said peptide or pharmaceutically acceptable salt in the second dosage (e.g., 700 or 800 μg/kg bodyweight).

Similar, the concentration of the third dosage may vary. Preferably, the third dosage is in the range 150-300 μg of said peptide or pharmacologically active salt per kg bodyweight, such as in the range 150-250 μg/kg bodyweight, such as in the range 150-200 μg/kg bodyweight, such as in the range 200-400 μg/kg bodyweight, such as in the range 200-300 μg/kg bodyweight. More preferably, the third dosage is 200 μg/kg bodyweight. Also more preferably, the third dosage is 300 μg/kg bodyweight.

The present invention features and contemplates any combination of the above-described or contemplated first, second and third dosages. For instance, the first dosage can be 200 μg/kg bodyweight, the second dosage can be 400 μg/kg bodyweight, and the third dosage can be 200 μg/kg bodyweight. For another instance, the first dosage can be 300 μg/kg bodyweight, the second dosage can be 600 μg/kg bodyweight, and the third dosage can be 300 μg/kg bodyweight.

In some cases, a method of the invention does not include any additional dosing of said peptide (e.g., AP214) or pharmaceutically acceptable salt thereof after 24 hours post the second dosage (or after 24 hours post cross clamp release).

Additional dosages may also be administered. In some cases, a method of the invention comprises additional dosing(s) after 24 hours post the second dosage (or after 24 hours post cross clamp release).

For instance, a method of the invention can comprise, in addition to the first, second and third dosages described or contemplated above, a fourth dosage at 12 hours post cross clamp release and a fifth dosage at 24 hours post cross clamp release. For another instance, a method of the invention can comprise, in addition to the first, second and third dosage described or contemplated above, a fourth dosage at 12 hours post cross clamp release, a fifth dosage at 24 hours post cross clamp release, and a sixth dosage at 48 hours post cross clamp release.

The drug concentration of each of the fourth, fifth, and sixth dosages may vary. For instance, the fourth, fifth, and sixth dosages can each independently be in the range of 150 μg/kg to 400 μg/kg bodyweight of said peptide or pharmacologically active salt thereof. Preferably, the fourth, fifth, and sixth dosages are each independently in the range 150-300 μg of said peptide or pharmacologically active salt per kg bodyweight, such as in the range 150-250 μg/kg bodyweight, such as in the range 150-200 μg/kg bodyweight, such as in the range 200-400 μg/kg bodyweight, such as in the range 200-300 μg/kg bodyweight. More preferably, the fourth, fifth, and sixth dosages are each 200 μg/kg bodyweight. Also more preferably, the fourth, fifth, and sixth dosages are each 300 μg/kg bodyweight.

The precise time of initiation of the first dosage may be further specified. Preferably, the initiation of the first dosage is at the time of skin incision. In the present context the term “at the time of skin incision” relates to the point in time the surgery is initiated by opening up the patient.

In one embodiment the initiation of administration of the first dosage is +/−20 minutes from initiation of surgery. In another embodiment the initiation of administration of the first dosage is +/−20 minutes from skin incision, such as +/−20 minutes, such as +/−15 minutes, such as +/−10 minutes, such as +/−5 minutes, or such as +/−1 minute. “+/−” means that the initiation of administration is either before or after the action at issue (e.g., skin incision or cross clamp release).

The precise time of initiation of the second dosage may also be further specified. Preferably, the initiation of administration of the second dosage is +/−20 from cross clamp release, such as +/−15 minutes from cross clamp release, such as +/−10 minutes from cross clamp release, such as +/−5 minutes from cross clamp release, or such as +/−1 minute from cross clamp release. More preferably, the initiation of administration of the second dosage is the time of cross clamp release. In the example sections results are shown where the second dosage is provided at the time of cross clamp release.

For example and without limitation, an (aortic) cross-clamp is a surgical instrument used in cardiac surgery to clamp the aorta and separate the systemic circulation from the outflow of the heart. An aortic cross clamping procedure serves, for example, in the repairing of coarctation of the aorta. The clamping of the aorta excludes the systemic circulation, by definition, thus causing an ischemia. Temporal ischemia of the kidney is frequently seen as a consequence of reduced blood pressure, hypovolemia, surgical interventions that involves reduction in renal and/or aortic blood flow, or associated with septicemia. This may result in ischemia-induce acute renal failure, which for a large fraction deteriorates into chronic renal failure. A common finding in the post ischemic phase is the development of urinary concentration defects with the formation of increased production of solute free urine.

Again, the precise time of initiation of the third dosage may be further specified. Preferably, the initiation of administration of the third dosage is 1-16 hours after cross clamp release, such as 1-8 hours after cross clamp release, such as 1-7 hours after cross clamp release, such as 3-10 hours after cross clamp release, such as 4-10 hours after cross clamp release, such as 5-10 hours after cross clamp release, such as 4-8 hours after cross clamp release, such as 5-7 hours after cross clamp release, or such as 6 hours after cross clamp release. Preferably, the third dosage is initiated 6 hours after the second dosage. More preferably, the third dosage is initiated 6 hours after cross clamp release. In the example section results are shown where the third dosage is provided 6 hours after cross clamp release.

Likewise, the precise time of initiation of the fourth, fifth and/or sixth dosages may also vary. Preferably, the initiation of administration of the fourth dosage is 4-12 hours after the third dosage, such as 4-8 hours after the third dosage, such as 6 hours after the third dosage. Preferably, the fourth dosage is initiated at 8-14 hours after cross clamp release. More preferably, the fourth dosage is initiated at 12 hours after cross clamp release.

Preferably, the initiation of administration of the fifth dosage is 4-24 hours after the fourth dosage, such as 8-16 hours after the fourth dosage, such as 12 hours after the fourth dosage. Preferably, the fifth dosage is initiated at 16-36 hours after cross clamp release. More preferably, the fifth dosage is initiated at 24 hours after cross clamp release.

Preferably, the initiation of administration of the sixth dosage is 12-36 hours after the fifth dosage, such as 16-32 hours after the fifth dosage, such as 24 hours after the fifth dosage. Preferably, the sixth dosage is initiated at 36-60 hours after cross clamp release. More preferably, the fourth dosage is initiated at 48 hours after cross clamp release.

In one embodiment, the subject does not receive further administration of said peptide (e.g., AP214) or a pharmaceutically acceptable salt thereof beyond the first, the second and the third dosage, such as within a period of 24 hours after cross clamp release, such as within a period of 48 hours after cross clamp release, such as within a period of 72 hours after cross clamp release, such as within a period of 96 hours after cross clamp release, such as within a period of one week after cross clamp release, such as within a period of two weeks after cross clamp release. The dosage regime of this embodiment can provides sufficient protection from AKI to the subject.

In another embodiment, the subject receives further administration of said peptide (e.g., AP214) or a pharmaceutically acceptable salt thereof beyond the first, the second and the third dosage, such as at 12 and 24 hours after cross clamp release, such as at 12, 24 and 48 hours after cross clamp release.

The present invention features and contemplates any combination of the above-described or contemplated timings for the first, second, third, fourth, fifth and sixth dosages. The present invention also features and contemplates any combination of the above-described or contemplated concentrations and timings for the first, second, third, fourth, fifth and sixth dosages. For instance, the first dosage can be 200 μg/kg bodyweight and initiated or administered at the time of skin incision, the second dosage can be 400 μg/kg bodyweight and initiated or administered at the cross clamp release, and the third dosage can be 200 μg/kg bodyweight and initiated or administered 6 hours after the cross clamp release. For another instance, the first dosage can be 300 μg/kg bodyweight and initiated or administered at the time of skin incision, the second dosage can be 600 μg/kg bodyweight and initiated or administered at the cross clamp release, and the third dosage can be 300 μg/kg bodyweight and initiated or administered 6 hours after the cross clamp release.

For yet another instance, the first dosage can be 300 μg/kg bodyweight and initiated or administered at the time of skin incision, the second dosage can be 600 μg/kg bodyweight and initiated or administered at the cross clamp release, the third dosage can be 300 μg/kg bodyweight and initiated or administered 6 hours after the cross clamp release, the fourth dosage can be 200 μg/kg bodyweight and initiated or administered 12 hours after the cross clamp release, and the fifth dosage can be 200 μg/kg bodyweight and initiated or administered 24 hours after the cross clamp release.

For yet another instance, the first dosage can be 300 μg/kg bodyweight and initiated or administered at the time of skin incision, the second dosage can be 600 μg/kg bodyweight and initiated or administered at the cross clamp release, the third dosage can be 300 μg/kg bodyweight and initiated or administered 6 hours after the cross clamp release, the fourth dosage can be 300 μg/kg bodyweight and initiated or administered 12 hours after the cross clamp release, the fifth dosage can be 300 μg/kg bodyweight and initiated or administered 24 hours after the cross clamp release, and the sixth dosage can be 300 μg/kg bodyweight and initiated or administered 48 hours after the cross clamp release.

The subject in need of the treatment protocol of the present invention may undergo different surgical procedures as well as non-surgical or other medical procedures. For instance, in any method described or contemplated herein, said surgery can be cardiac or vascular surgery. For another instance, in any method described or contemplated herein, the subject is undergoing cardiac surgery with cardiopulmonary bypass, and/or an aortic cross clamping procedure. For yet another instance, in any method described or contemplated herein, the subject is undergoing aortic surgery, such as coarctation. For yet another instance, in any method described or contemplated herein, the subject is undergoing trauma surgery, transplant surgery, or pediatric surgery. For yet another instance, in any method described or contemplated herein, the subject is undergoing percutaneous coronary intervention (PCI). For still another instance, in any method described or contemplated herein, such subjects are at risk of developing AKI and may therefore be in need of the treatment protocol according to the present invention.

Any method described or contemplated herein can be used to prevent or reduce AKI associated with surgeries as well as non-surgical or other medical procedures comprising cross clamping.

The subject according to the present invention, or in any method described or contemplated herein, is preferably a human such as a female or male human being.

The dosage regime according to the present invention may be administered by different routes to the subject. For instance, in any method described or contemplated herein, the route of administration is intravenous. Intravenous injection has been used in the examples described below.

The pharmaceutical composition may comprise further components. Thus, in an embodiment the pharmaceutical composition according to the present invention further comprises one or more pharmaceutical carriers. In another embodiment, the pharmaceutical composition further comprises one or more pharmaceutically acceptable excipients. The compositions may be formulated according to conventional pharmaceutical practice, see, e.g., “Remington: The science and practice of pharmacy” 20th ed. Mack Publishing, Easton Pa., 2000; and “Encyclopedia of Pharmaceutical Technology”, edited by Swarbrick, J. & J. C. Boylan, Marcel Dekker, Inc., New York, 1988. Official pharmacopeias such as the British Pharmacopeia, the United States of America Pharmacopeia and the European Pharmacopeia set standards for well-known pharmaceutically acceptable excipients.

Further studies have also surprisingly shown that the control of infusion rate of said peptide (e.g., AP214) can reduce side effects of said peptide. Thus, the first dosage preferably is administered to the patient over a period of 5-20 minutes, such as 5-15 minutes, such as 5-10 minutes, such as 10-15 minutes, such as 10-20 minutes, such as 15-20 minutes, such as 10 minutes, such as 15 minutes, or such as 20 minutes. The second dosage preferably is administered to the patient over a period of 5-20 minutes, such as 5-15 minutes, such as 5-10 minutes, such as 10-15 minutes, such as 10-20 minutes, such as 15-20 minutes, such as 10 minutes, such as 15 minutes, or such as 20 minutes. The third dosage preferably is administered to the patient over a period of 5-20 minutes, such as 5-15 minutes, such as 5-10 minutes, such as 10-15 minutes, such as 10-20 minutes, such as 15-20 minutes, such as 10 minutes, such as 15 minutes, or such as 20 minutes.

Similarly, the fourth dosage preferably is administered to the patient over a period of 5-20 minutes, such as 5-15 minutes, such as 5-10 minutes, such as 10-15 minutes, such as 10-20 minutes, such as 15-20 minutes, such as 10 minutes, such as 15 minutes, or such as 20 minutes. The fifth dosage preferably is administered to the patient over a period of 5-20 minutes, such as 5-15 minutes, such as 5-10 minutes, such as 10-15 minutes, such as 10-20 minutes, such as 15-20 minutes, such as 10 minutes, such as 15 minutes, or such as 20 minutes. The sixth dosage preferably is administered to the patient over a period of 5-20 minutes, such as 5-15 minutes, such as 5-10 minutes, such as 10-15 minutes, such as 10-20 minutes, such as 15-20 minutes, such as 10 minutes, such as 15 minutes, or such as 20 minutes.

Clinical tests have shown that infusing said peptide (e.g., AP214) at faster rates can result in more severe side to the patient. This is in contrast to previous studies conducted on pigs (see Matthew N. Simmons et al, supra).

The present invention features and contemplates any combination of the above-described or contemplated concentrations, timings, and infusion rates for the first, second, third, fourth, fifth and sixth dosages. For instance, the first dosage can be 200 μg/kg bodyweight and initiated or administered at the time of skin incision, the second dosage can be 400 μg/kg bodyweight and initiated or administered at the cross clamp release, and the third dosage can be 200 μg/kg bodyweight and initiated or administered 6 hours after the cross clamp release, wherein each of the first, second and third dosages is administered over a period of 10 minutes. Each of these dosages can also be administered for at least 10 minutes or any other suitable period as described or contemplated above.

For another instance, the first dosage can be 300 μg/kg bodyweight and initiated or administered at the time of skin incision, the second dosage can be 600 μg/kg bodyweight and initiated or administered at the cross clamp release, and the third dosage can be 300 μg/kg bodyweight and initiated or administered 6 hours after the cross clamp release, wherein each of the first, second and third dosages is administered over a period of 10 minutes. Each of these dosages can also be administered for at least 10 minutes or any other suitable period as described or contemplated above.

For yet another instance, the first dosage can be 300 μg/kg bodyweight and initiated or administered at the time of skin incision, the second dosage can be 600 μg/kg bodyweight and initiated or administered at the cross clamp release, the third dosage can be 300 μg/kg bodyweight and initiated or administered 6 hours after the cross clamp release, the fourth dosage can be 200 μg/kg bodyweight and initiated or administered 12 hours after the cross clamp release, and the fifth dosage can be 200 μg/kg bodyweight and initiated or administered 24 hours after the cross clamp release, wherein each of the first, second, third, fourth and fifth dosages is administered over a period of 10 minutes. Each of these dosages can also be administered for at least 10 minutes or any other suitable period as described or contemplated above.

For yet another instance, the first dosage can be 300 μg/kg bodyweight and initiated or administered at the time of skin incision, the second dosage can be 600 μg/kg bodyweight and initiated or administered at the cross clamp release, the third dosage can be 300 μg/kg bodyweight and initiated or administered 6 hours after the cross clamp release, the fourth dosage can be 300 μg/kg bodyweight and initiated or administered 12 hours after the cross clamp release, the fifth dosage can be 300 μg/kg bodyweight and initiated or administered 24 hours after the cross clamp release, and the sixth dosage can be 300 μg/kg bodyweight and initiated or administered 48 hours after the cross clamp release, wherein each of the first, second, third, fourth, fifth, and sixth dosages is administered over a period of 10 minutes. Each of these dosages can also be administered for at least 10 minutes or any other suitable period as described or contemplated above.

Where a subject undergoes PCI, two doses can be sufficient for preventing or reducing AKI. The first dosage can be administered at the initiation of the procedure, and the second dosage can be administered from 2 to 6 hours after the initiation of the procedure. Preferably, the second dosage is administered from 3 to 4 hours after the initiation of the procedure. The concentration of each of the first and second dosages may vary. Preferably, each of the first and second dosages is independently in the range of 200-600 μg per kg bodyweight of said peptide (e.g., AP214) or a pharmacologically active salt thereof, such as in the range of 300-600 μg/kg bodyweight, such as in the range of 400-600 μg/kg bodyweight, such as in the range of 500-600 μg/kg bodyweight, such as in the range 200-300 μg/kg bodyweight, such as in the range of 200-400 μg/kg bodyweight, such as in the range of 200-500 μg/kg bodyweight, such as in the range 300-400 μg/kg bodyweight, such as in the range 300-500 μg/kg bodyweight, such as in the range 400-500 μg/kg bodyweight. More than 600 μg/kg bodyweight of said peptide or its pharmaceutically acceptable salt (e.g., 700 or 800 μg/kg bodyweight) can also be used for each dosage. The administration of each dosage can last for any suitable period described or contemplated herein, such as over a period of 10 minutes.

Any method described or contemplated herein can also be used to prevent or reduce AKI in a subject undergoing a surgery or medical procedure that does not require cross clamping. The second dosage in such methods can be administered or initiated 2-4 hours after the skin incision or the initiation of the medical procedure. For example, the second dosage can be administered or initiated at 2 hours after the skin incision or the initiation of the medical procedure. The timing of the third dosage in such methods, as well as the timings of the fourth, fifth and/or sixth dosages when used, can be measured from the administration of the second dosage, in lieu of cross clamp release. Therefore, any concentrations, timings, and infusion rates for the first, second, third, fourth, fifth and sixth dosages described or contemplated hereinabove, and any combination thereof, can be used in such methods, except that the timing of the second dosage is as described in this paragraph and that the timings of the third, fourth, fifth and sixth dosages are measured from the administration of the second dosage, in lieu of cross clamp release.

Moreover, any method described or contemplated herein can be used to prevent or reduce inflammatory conditions or reactions, or other kidney injuries, associated with surgeries or non-surgical procedures described or contemplated herein.

Yet another aspect of the present invention relates to a pharmaceutical composition comprising a peptide comprising the amino acid sequence set forth in SEQ ID: NO 1 (e.g. AP214) or a pharmacologically acceptable salt thereof for use in the prevention or reduction of Acute Kidney Injury (AKI) in a subject undergoing surgery comprising cross clamping, wherein said compound is provided in a dosage scheme comprising
administering to the subject a first dosage of 150 μg/kg to 400 μg/kg bodyweight of a peptide comprising the amino acid sequence set forth in SEQ ID: NO 1 (e.g. AP214) or a pharmacologically acceptable salt thereof, said administration being initiated at the initiation of said surgery (e.g., prior to or at skin incision);
administering to the subject a second dosage of 150 to 600 μg/kg bodyweight of said peptide or pharmacologically active salt thereof, administration of said second dosage being initiated prior to or at cross clamp release; and
administering to the subject a third dosage of 150 to 400 μg/kg bodyweight of said peptide or pharmacologically active salt thereof, administration of said third dosage being initiated 1-24 hours after cross clamp release (e.g., at 6 hours after cross clamp release or the second dosage).

Any above-described or contemplated dosages, timings, and infusion rates for the first, second, third, fourth, fifth and sixth dosages, or any combinations thereof, can be employed in this aspect of the invention.

It should be noted that embodiments and features described in the context of one of the aspects of the present invention also apply to the other aspects of the invention.

The invention will now be described in further details in the following non-limiting examples.

### EXAMPLES

### Example 1

Comparison in the prevention of AKI by different dosage regimes of AP214.

Study Design

Each of 12 patients undergoing cardiac surgery (the AP214 group) was dosed with 600 μg/kg AP214: 200 μg/kg at skin incision, 200 μg/kg at cross clamp release, and 200 μg/kg 6-hour after cross clamp release. Each dosage was provided over a period of 10 minutes. The placebo group included 13 patients undergoing cardiac surgery without AP214 infusion.

Objectives
trial objective was to assess the effect of AP214 on
   1) changes in serum creatinine, cystatin-C and carbamade,
   2) on eGFR and
   3) on the development of post-surgical acute kidney injury (AKI)
AKI also assessed (post-hoc analysis) by AKIN and RIFLE

Results

Impact on eGFR and Serum Creatinine:

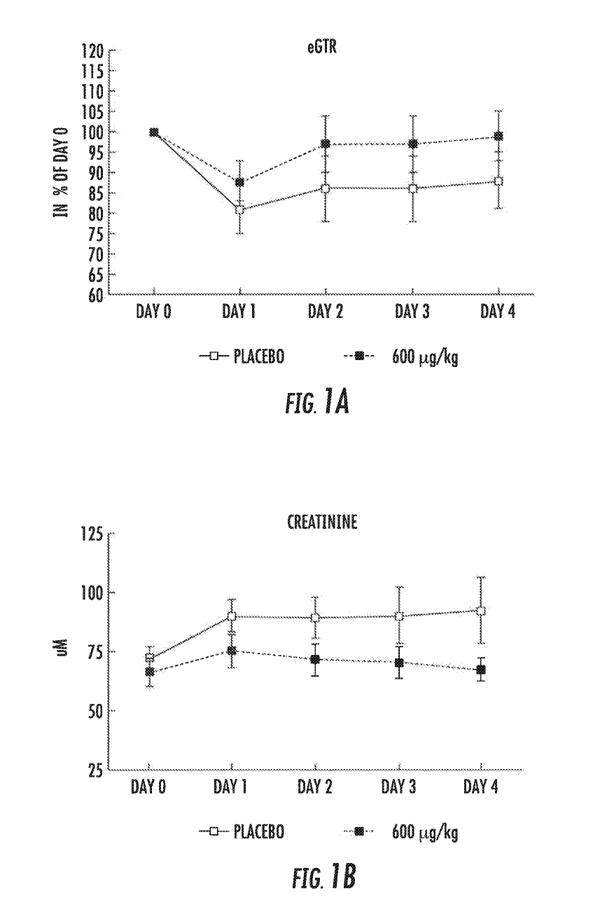

AP214 at 600 μg/kg bodyweight (3×200 μg/kg bodyweight) prevented a decrease in eGFR and increase in serum creatinine as shown in FIGS. 1a and 1b, respectively.

Impact on Cystatin C and Carbamide:

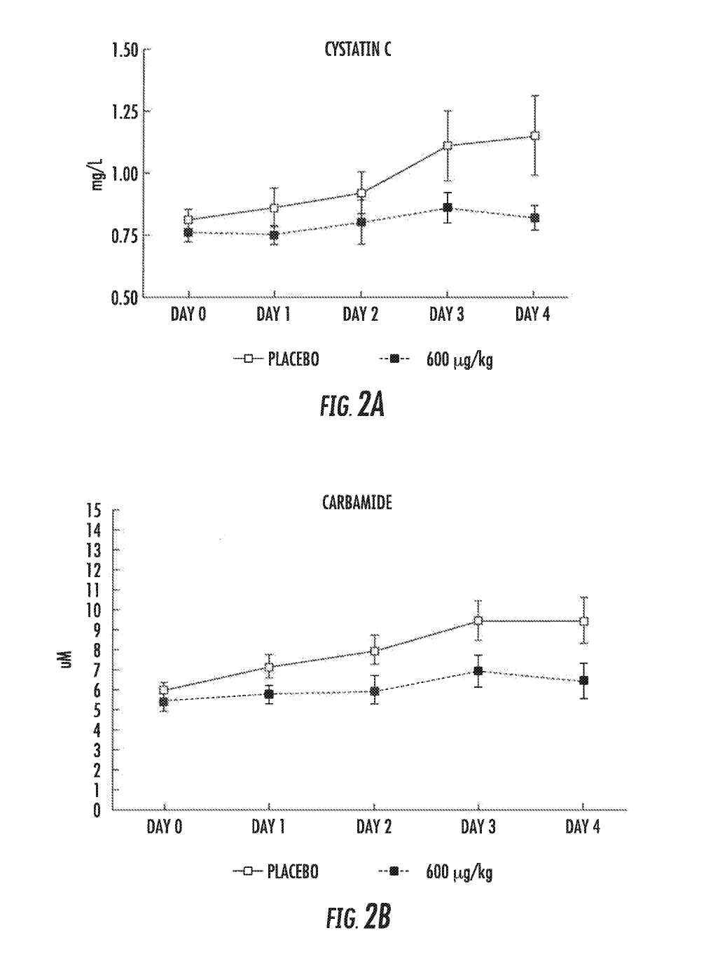

AP214 at 600 μg/kg bodyweight prevented an increase in cystatin C and carbamide as shown in FIGS. 2a and 2b, respectively.

Prevention in the Development of AKI:

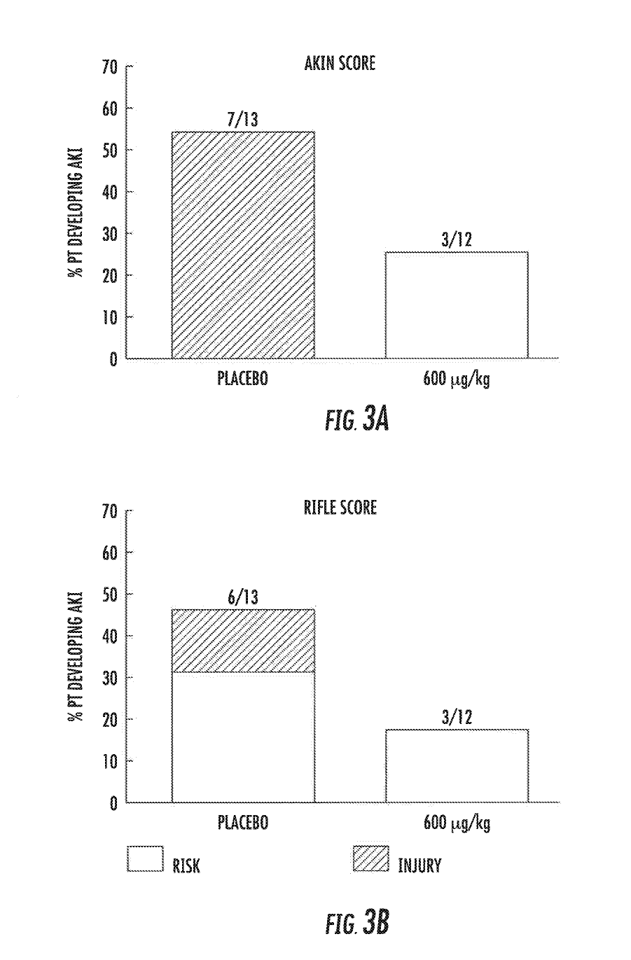

Three different definitions of AKI were tested:
1) 3 patients in the AP214 group developed AKI, in contrast to 7 patients in the placebo group who developed AKI. See FIG. 3. AKI is defined as in the CS005 protocol:
   An absolute increase in serum creatinine of more than or equal to 26.4 μmol/l (0.3 mg/dl) or
   A percentage increase in serum creatinine of more than or equal to 50% (1.5-fold from baseline) from Day 0-14 and/or
   Urine output less than 0.5 mL/kg per hour for more than 6 hours.
2) AKI according to the AKIN score as presented in FIG. 3a, and
3) AKI according to the RIFLE score as presented in FIG. 3b.

Conclusion

AP214 at 600 μg/kg bodyweight (3×200 μg/kg bodyweight) demonstrated prevention in the development of AKI according to the RIFLE score and AKIN scores (FIG. 3). In contrast, AP214 at 150 μg/kg bodyweight (3×50 μg/kg bodyweight according to the same dosing scheme) showed no prevention of AKI as compared to the placebo.

### Example 2

This trial (The CS007 trial) was designed to study both short and long term efficacy signals after AP214 treatment.

Study Design

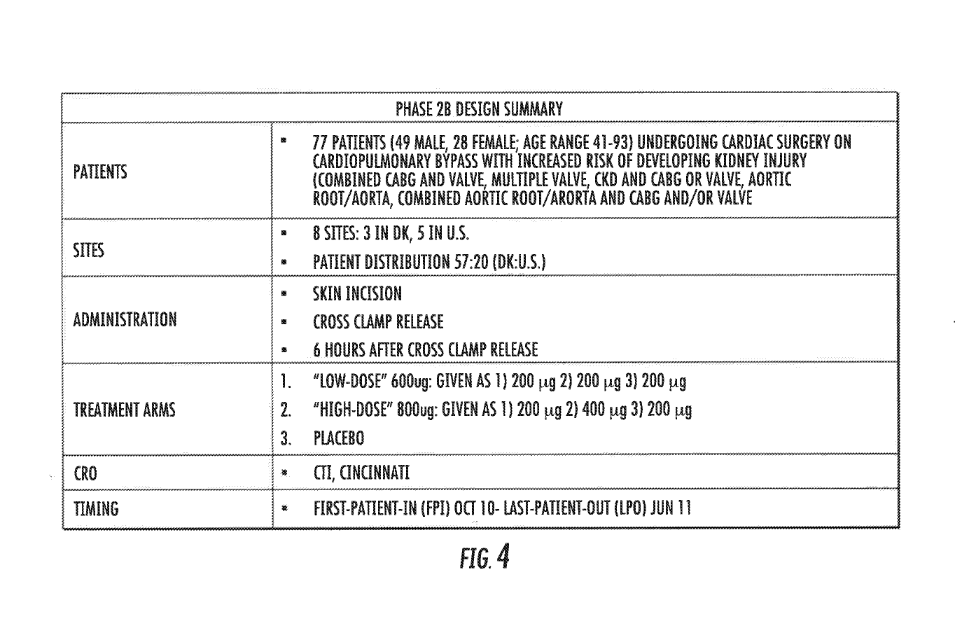

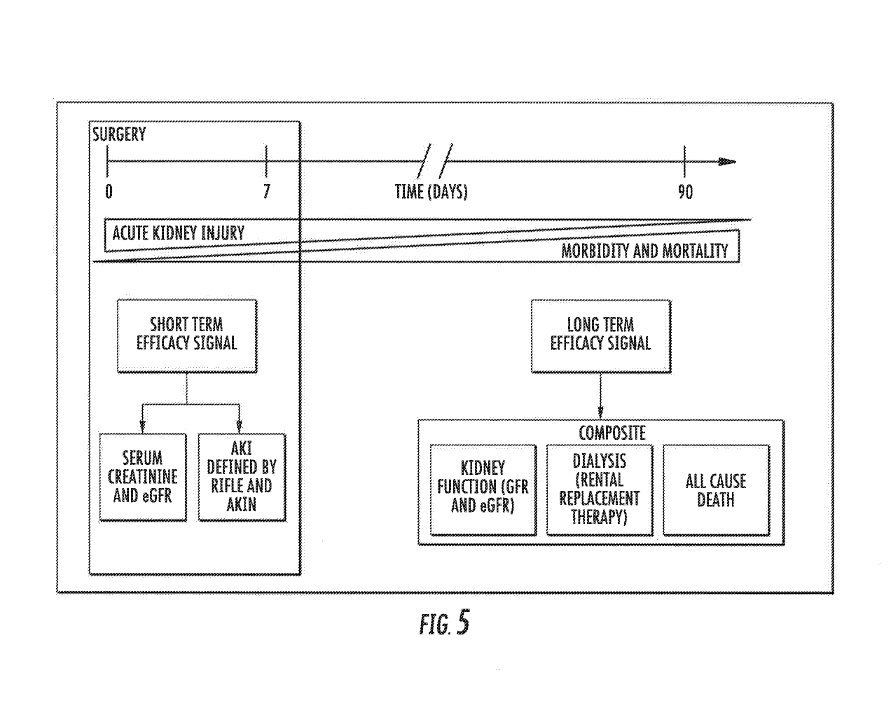

FIG. 4 shows the study design of the CS007 trial where also the two different dosage regimes of AP214 are described. The CS007 trial was designed to study both short and long term efficacy signals (FIG. 5).

Primary Aims
Safety: Safety and tolerability vs. placebo
Efficacy: Max post-operative change in absolute values of SCr compared to baseline within the first 7 days after surgery or until discharge from hospital, whichever comes first vs. placebo.

Secondary Aims
Composite:
   Assess the proportion of patients reaching the composite endpoint of death, need for RRT or a 25% reduction in renal function over a 90 day post-operative period vs. placebo.
AKIN: Assess post-operative incidence of AKI within 48 hours post-surgery
RIFLE: Assess post-operative incidence of AKI within first 7 days post-surgery.
SCr: Changes between surgery and postoperative day 7.
GFR: Changes at day 90 compared to baseline.
eGFR: Changes between surgery and postoperative day 7

Results on Short Term Efficacy Signals (Day 0-7)

Patient groups treated with the 600 μg/kg (3×200 μg/kg) and 800 μg/kg (1×200, 1×400, 1×200 μg/kg) doses of AP214, both of which were administered based on the dosing scheme described in Example 1, demonstrated decreasing average serum creatinine values over time (through 168 hours post surgery). Each dosage was provided over a period of 10 minutes. As used throughout this disclosure and unless specified otherwise, “μg/kg” refers to μg AP214 per kg bodyweight of the patient being treated.

Compared to placebo, the population treated with the 800 μg/kg dose of AP214 displayed lesser degrees of acute kidney injury.

Conclusion on Short Term Efficacy Signals (Day 0-7)

Both Doses of AP214 Demonstrated Short Term Efficacy Signals
Average serum creatinine and eGFR: Both patient groups treated with AP214 showed decreased average serum creatinine and increased kidney function as measured by estimated GFR in the seven day post-operative period.
Population distribution of extreme serum creatinine values: Both patient groups treated with AP214 showed a reduction in serum creatinine values at the highest quartile suggesting that AP214 helps blunt the incidence of AKI.
Patients scored as AKI: The patient group treated with 800 μg/kg of AP214 showed a decrease in patients scored as AKI by AKIN and RIFLE criteria.

Results on Long Term Efficacy Signals

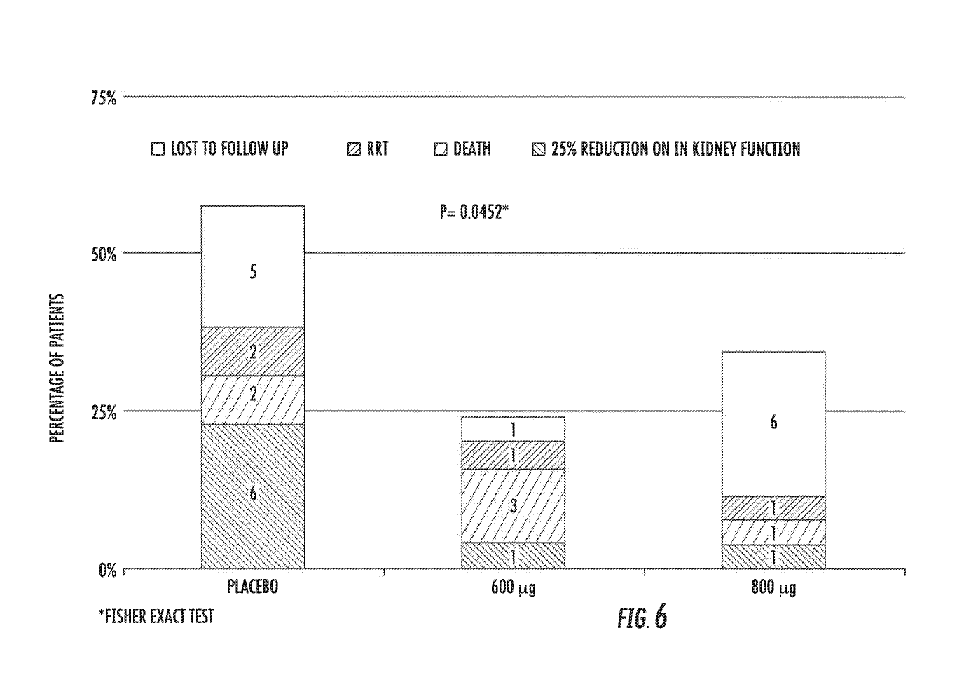

Both the 600 and 800 μg/kg doses showed a reduction in outcomes with the 600 μg/kg dosage showing statistical significance as measured by the composite endpoint (FIG. 6).

GFR Results

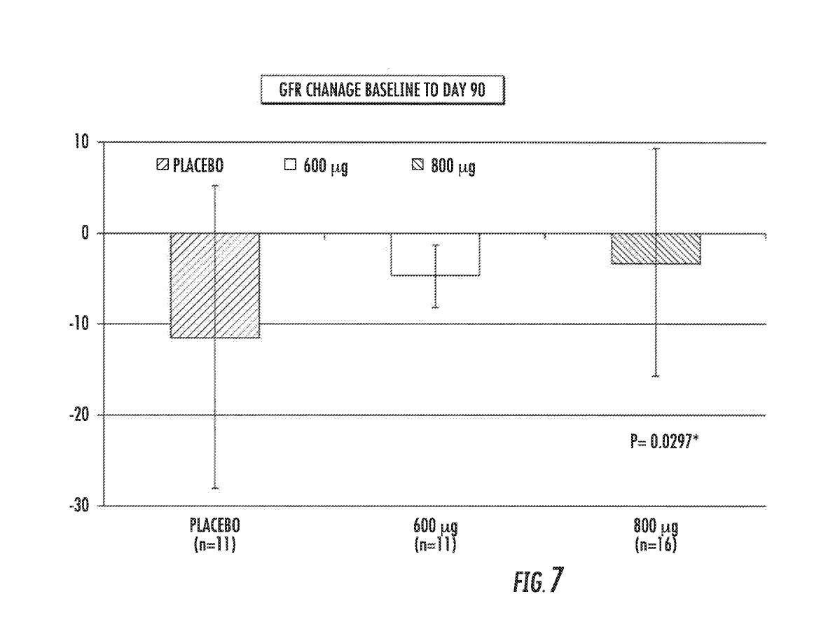

Significantly lower GFR change (reduction) at Day 90 compared to baseline for AP214 800 μg/kg dose vs. placebo (FIG. 7). GFR can be considered the most valid measurement of kidney function and superior to any judgement of serum creatinine changes or eGFR calculations. For practical reasons, this measurement was only done at Danish sites.

Conclusion on Long Term Efficacy Signals

AP214 showed significant effect on the composite endpoint (all cause death, RRT, kidney function). AP214 also showed significant effect on GFR measurements—a precise means to assessing renal function—at Day 90.

Overall Conclusion
AP214 is safe and well tolerated;
AP214 demonstrated a reduction in Acute Kidney Injury (RIFLE, AKIN scores);
Protective effect of AP214 demonstrated by serum creatinine and GFR;
AP214 showed significant effect on the composite endpoint (all cause death, RRT, kidney function);
AP214 showed significant effect on GFR measurements—a precise means to assessing renal function—at Day 90;

Thus, the presented data shows that AP214 is safe, well tolerated and demonstrated efficacy signals.

Moreover, in the same double-blind study described in this Example, patients undergoing cardiac surgery on cardiopulmonary bypass (CPB) were randomized to placebo (PBO; n=26), AP214 given at either 600 μg/kg (n=25), or 800 μg/kg (n=26), divided into 3 bolus infusions at the predetermined intervals as described above. AKI was determined according to AKIN and RIFLE scores.

The majority of the patients (53%) underwent combined coronary artery bypass grafting (CABG) and valve surgery. In the PBO arm, the group with combined CABG and valve surgery had high incidence of AKI. Treatment with 800 μg/kg of AP214 resulted in a numerically lower incidence of AKI, compared with PBO, for the following surgery types: (1) combined CABG and valve surgery, (2) multiple valve surgery, and (3) chronic kidney disease and CABG (or valve surgery).

### Example 3

### Evaluation of Rate of Infusion of AP214 (CS002)

To establish a suitable rate of infusion of AP214 a set of patient trials were performed.

Test Groups

Group 1 (n=40)

AP214 isotonic solution single ascending doses (25, 50 and 100 μg/kg) for intravenous infusion administered over 10 minutes, or placebo (saline infusion).

Group 2 (n=6)

AP214 isotonic solution single doses (100 μg/kg over 1 minute; 100 μg/kg over 30 seconds; 200 μg/kg over 30 seconds) for intravenous infusion.

Results

A large number of adverse events were reported, which was considered relating to short infusion times. Most frequently reported were ear discomfort, nausea, feeling cold, headache, paresthesia, erythema (all 6 subjects) and hot flush. One subject experienced 8 episodes of vomiting. The subjects who received 200 μg/kg AP214 over 30 seconds had more adverse events than those who received 100 μg/kg over 30 sec and 1 minute. Subjects receiving slower infusion rate (100 μg/kg AP214 over 10 minutes) did not show these adverse effects.

### CONCLUSION

These finding showed that AP214 was not well tolerated when administered with fast infusion rates, whereas administration with slower infusion rates (e.g., 10 minutes) was better tolerated.

What is claimed is:

1. A method of preventing or reducing Acute Kidney Injury (AKI) in a subject undergoing surgery comprising cross clamping, wherein the method comprises:
I. administering to the subject a first dosage of 150 μg/kg to 400 μg/kg bodyweight of a peptide comprising the amino acid sequence set forth in SEQ ID: NO 1 or a pharmacologically acceptable salt thereof, said administration being initiated before said surgery;
II. administering to the subject a second dosage of 150 to 600 μg/kg bodyweight of said peptide or pharmacologically active salt thereof, administration of said second dosage being initiated at cross clamp release; and
III. administering to the subject a third dosage of 150 to 400 μg/kg bodyweight of said peptide or pharmacologically active salt thereof, administration of said third dosage being initiated 1-24 hours after cross clamp release.

2. The method according to claim 1, wherein said peptide or pharmacologically acceptable salt is 19 amino acid residues in length.

3. The method according to claim 1, wherein the dosages are administered with equal amounts or substantially equal amounts of said peptide or pharmacologically active salt.

4. The method according to claim 1, wherein the first dosage is in a range of 150-300 μg of said peptide or pharmacologically active salt per kg bodyweight, and the second dosage is in a range of 200-600 μg of said peptide or pharmacologically active salt per kg bodyweight, and the third dosage is in the range 150-300 μg of said peptide or pharmacologically active salt per kg bodyweight.

5. The method according to claim 4, wherein the initiation of the first dosage is +/−5 minutes from skin incision, the initiation of the second dosage is +/−5 minutes from said cross clamp release, and the initiation of the third dosage is 4-10 hours after said cross clamp release.

6. The method according to claim 4, wherein the initiation of the first dosage is at the time of skin incision, and the initiation of the second dosage is at said cross clamp release, and the initiation of the third dosage is at 6 hour after said cross clamp release.

7. The method according to claim 4, wherein said first dosage, second dosage and third dosage is each administered to said subject over a period of from 5 to 15 minutes.

8. The method according to claim 4, wherein said first dosage, second dosage and third dosage is each administered to said subject over a period of 10 minutes.

9. The method according to claim 6, wherein said first dosage, second dosage and third dosage is each administered to said subject over a period of from 5 to 15 minutes.

10. The method according to claim 6, wherein said first dosage, second dosage and third dosage is each administered to said subject over a period of 10 minutes.

11. The method according to claim 1, wherein said peptide or pharmacologically active salt is AP214 or a pharmacologically active salt thereof.

12. The method according to claim 11, wherein the first dosage is in a range of 150-300 μg of said AP214 or pharmacologically active salt per kg bodyweight, and the second dosage is in a range of 200-600 μg of said AP214 or pharmacologically active salt per kg bodyweight, and the third dosage is in the range 150-300 μg of said AP214 or pharmacologically active salt per kg bodyweight.

13. The method according to claim 12, wherein the initiation of the first dosage is +/−5 minutes from skin incision, the initiation of the second dosage is +/−5 minutes from said cross clamp release, and the initiation of the third dosage is 4-10 hours after said cross clamp release.

14. The method according to claim 12, wherein the initiation of the first dosage is at the time of skin incision, and the initiation of the second dosage is at said cross clamp release, and the initiation of the third dosage is at 6 hour after said cross damp release.

15. The method according to claim 12, wherein said first dosage, second dosage and third dosage is each administered to said subject over a period of from 5 to 15 minutes.

16. The method according to claim 12, wherein said first dosage, second dosage and third dosage is each administered to said subject over a period of 10 minutes.

17. The method according to claim 14, wherein said first dosage, second dosage and third dosage is each administered to said subject over a period of from 5 to 15 minutes.

18. The method of claim 14, wherein said first dosage, second dosage and third dosage is each administered to said subject over a period of 10 minutes.

19. The method according to claim 1, wherein the subject is undergoing cardiovascular surgery.

20. The method according to claim 1, wherein the subject is undergoing cardiovascular surgery with cardiopulmonary bypass or an aortic cross clamping procedure.

21. The method according to claim 1, wherein the subject is undergoing an aortic surgery.


SEQUENCE LISTING (7 sequences)

SEQ ID NO: 1 - DNA, 25 bp, Artificial Sequence
  Description of Artificial Sequence Synthetic oligonucleotide
gggatggggtgggtggccagcgatt

SEQ ID NO: 2 - DNA, 25 bp, Artificial Sequence
  Description of Artificial Sequence Synthetic oligonucleotide
ttaggggtggtgtggttggcaattc

SEQ ID NO: 3 - DNA, 72 bp, Artificial Sequence
  Description of Artificial Sequence Synthetic oligonucleotide
gggaccatggaataaacgctgaannnnnnnnnnnnnnnnnnnnnnnnnatcagcaggaagctcgagacaggc

SEQ ID NO: 4 - DNA, 23 bp, Artificial Sequence
  Description of Artificial Sequence Synthetic primer
gggaccatggaataaacgctgaa

SEQ ID NO: 5 - DNA, 24 bp, Artificial Sequence
  Description of Artificial Sequence Synthetic primer
gcctgtctcgagcttcctgctgat

SEQ ID NO: 6 - DNA, 59 bp, Artificial Sequence
  Description of Artificial Sequence Synthetic primer
ccatctcatccctgcgtgtctccgactcagatgatagggaccatggaataaacgctgaa

SEQ ID NO: 7 - DNA, 40 bp, Artificial Sequence
  Description of Artificial Sequence Synthetic primer
cctctctatgggcagtcggtgatgcctgtctcgagcttcc